(19) United States
(12) Patent Application Publication
(10) Pub. No.: US 2007/0027067 A1
(43) Pub. Date: Feb. 1, 2007

(54) Transition-state Inhibitors of Pin1, alpha-Ketoamide-containing peptidomimetics, and synthesis thereof

(76) Inventor: Felicia Etzkorn, Blacksburg, VA (US)

Correspondence Address: WHITHAM, CURTIS & CHRISTOFFERSON & COOK, P.C., 11491 SUNSET HILLS ROAD, SUITE 340, RESTON, VA, 20190, US

(21) Appl. No.: 11/383,085

(22) Filed: May 12, 2006

Related U.S. Application Data
(60) Provisional application No. 60/680,518, filed on May 13, 2005.

(51) Int. Cl. A61K 38/16; A61K 31/675; C07F 9/547

(52) U.S. Cl. 514/7; 514/91; 548/413

(57) ABSTRACT

Novel α-ketoamide-containing peptidomimetics are provided, such as peptidomimetics containing an α-ketoamide Ser-Pro dipeptide analogue. The α-ketoamide is preferably incorporated into another molecule as a Pin1 inhibitor (such as a pentapeptide analogue Ac-Phe-Tyr-pSer-Pro-Arg-NH₂).

### RELATED APPLICATION

This application claims benefit of U.S. provisional application Ser. No. 60/680,518 filed May 13, 2005 titled “Synthesis of Transition-State Inhibitors of Pin1.”

The invention was made using support from the National Institutes of Health under Grant R01 GM63271, and the U.S. Government may have certain rights in the invention.

### FIELD OF THE INVENTION

This invention relates to the design and synthesis of compounds that are Pin1 inhibitors.

### BACKGROUND OF THE INVENTION

Conventionally, most cell cycle inhibitors have targeted kinases or phosphatases, of which there are very many. Because of the existence of so many similar targets, attaining specificity (namely, enzyme specificity) by targeting kinases or phosphatases is difficult.

More recently, certain small molecules have begun to be designed to mimic peptides in order to determine which amide form is critical to the biological function of peptidyl-prolyl isomerases (PPIases), such as cyclophilin, with particular attention to (Z)-alkene mimics. Hart and Etzkom (2000); Hart, Trindle and Etzkom (2001). In about April 2002, drug design to stop the cancer cell cycle was under consideration, and the cell-cycle-regulating enzyme, Pin1, was targeted, with an eye towards anticancer activity. (Virginia Tech Press release dated Apr. 10, 2002, “Chemists Explore the Shape of the Key that Signals Cell Division in Cancer Cells). At that time, the single known inhibitor of Pin1 was a natural product, juglone, that is not specific for PinI and is a poor inhibitor. (Hennig, L., Christner, C., Kipping, M., Schelbert, B., Rucknagel, K. P., Grabley, S., Kullertz, G., and Fischer, G. (1998), Selective inactivation of parvulin-like peptidyl-prolyl cis/trans isomerases by juglone, Biochemistry 37, 5953-5960.)

Regulation of the cell cycle is of fundamental significance in developmental biology and gives rise to cancer when it goes awry. The enzyme Pin1 is a phosphorylation-dependent peptidyl-prolyl isomerase (PPIase) enzyme thought to regulate mitosis via cis-trans isomerization of phosphoSer-Pro amide bonds in a variety of cell cycle proteins. (Lu, K. P., Hanes, S. D., and Hunter, T. (1996), A human peptidyl-prolyl isomerase essential for regulation of mitosis, Nature 380, 544-547; Yaffe, M. B., Schutkowski, M., Shen, M., Zhou, X. Z., Stukenberg, P. T., Rahfeld, J.-U., Xu, J., Kuang, J., Kirshcner, M. W., Fischer, G., Cantley, L. C., and Lu, K. P., Science 278 (1997) 1957.) In particular, Pin1 has been shown to bind phosphoSer-Pro epitopes in Cdc25 phosphatase, a key regulator of the Cdc2/cyclinB complex. (King, R. W., Jackson, P. K., and Kirschner, M. W., Cell 79 (1994) 563.) The central role Pin1 plays in the cell cycle makes Pin1 an interesting target for inhibition, both for potential anti-cancer activity and for elucidation of the mechanism of mitosis regulation. It has been proposed (Shen, M., Stukenberg, P. T., Kirschner, M. W., and Lu, K. P., Genes Dev. 12 (1998) 706) that Pin1 recognition of the phosphoSer-Pro amide bond acts as a conformational switch in the cell cycle.

Pin1 is unique among PPIases. (Lu, K. P., Hanes, S. D., Hunter, T., Nature 1996, 380, 544-547.) Pin1 regulates the entry into mitosis by catalyzing the conformational change on Cdc25 which regulates Cdc2, the central mitotic kinase. (Stukenberg, P. T., Kirschner, M. W., Pin1 Acts Catalytically to Promote a Conformational Change in Cdc26, Mol. Cell 2001, 7, 1071-1083.) Pin1 also regulates the activity of Cyclin D1, another cell-cycle protein that is active in G1. (Wulf, G. M., Liou, Y. C., Ryo, A. L., et al., Role of Pin1 in the Regulation of p53 Stability and p21 Transactivation, and Cell Cycle Checkpoints in Response to DNA Damage, J Biol. Chem., 2002, 277, 47976-47979.) Pin1 has two domains: WW domain and PPIase domain. Both of these domains recognize the phosphoserine-proline or phsophothreonine-proline bonds present in mitotic phosphoproteins. (Yaffe et al., supra.)

For additional background regarding Pin1, see also U.S. Pat. No. 6,596,848 issued Jul. 22, 2003 to Hunter et al. (Salk Institute) for “Antibodies to NIMA interacting proteins”, which is herein incorporated by reference.

Preference for phosphorylated substrates by Pin1 has been clearly demonstrated (Yaffe, supra), with the central dipeptide phosphoSer-Pro as the primary recognition element. Successful laboratory work has been accomplished using a (Z)-alkene amide bond isostere to mimic the Ala-cis-Pro amide bond for the inhibition of the PPIase cyclophilin, which then led to design of an analogous inhibitor based on a substrate for Pin1. Hart, S. A., Sabat, M., and Etzkom, F. A., J. Org. Chem. 63 (1998) 7580; Hart, S. A., and Etzkom, F. A., J. Org. Chem. 64 (1999) 2298. Synthesis of the Boc-Ser-Ψ[(Z)CH=C]-Pro mimic proceeded with regio- and enantio-selectivity through a [2,3]-sigmatropic rearrangement. Wang, X. J., Hart, S. A., Xu, B., Mason, M. D., Goodell, J. R., and Etzkom, F. A. (2003), Serine-cis-proline and Serine-trans-proline Isosteres: Stereoselective Synthesis of (Z)- and (E)-Alkene Mimics by Still-Wittig and Ireland-Claisen Rearrangements, J. Org. Chem. 68, 2343-2349.

The possibility of Pin1 activity led to interest and work on certain alkene mimics. (Wang, supra); Wang, X. J., Xu, B., Mullins, A. B., Neiler, F. K., and Etzkom, F. A. (2004), Conformationally Locked Isostere of PhosphoSer-cis-Pro Inhibits Pin1 23-Fold Better than PhosphoSer-trans-Pro Isostere, J. Am. Chem. Soc. 126, 15533-15542.

However, relatively few inhibitors of Pin1 are known, and Pin1 inhibitors with greater inhibitory activity would be desirable for medical applications. (Hennig, supra); Uchida, T., Takamiya, M., Takahashi, M., Miyashita, H., Ikeda, H., Terada, T., Matsuo, Y., Shirouzu, M., Yokoyama, S., Fujimori, F., and Hunter, T. (2003), Pin1 and Par14 peptidyl prolyl isomerase inhibitors block cell proliferation, Chem. Biol. 10, 15-24.1.) Nanomolar peptide inhibitors of Pin1 have been discovered recently from combinatorial libraries. (Wildemann, D.; Erdmann, F.; Alvarez, B. H.; Stoller, G.; Zhou, X. Z.; Fanghanel, J.; Schutkowski, M.; Lu, K. P.; Fischer, G., (2006) Nanomolar inhibitors of the peptidyl prolyl cis/trans isomerase Pin1 from combinatorial peptide libraries, J Med Chem, 49, 2147-50.)

The reversible phosphorylation of proteins is the most important post-translational modification that occurs in the cell. It is also the most efficient and versatile signal of intermolecular communication. As a result, many drug targets show high-affinity interactions with phosphorylated molecules, while their unphosphorylated counterparts do not bind well to the targets. However, there is a problem for these phosphorylated molecules: unprotected phosphorylated compounds are not effective at penetrating cell membranes, thus are not bioactive because of the negative charges on phosphate groups. One general approach to this problem involves masking the phosphate in a form that neutralizes their negative charges. Among the reversibly masking phosphate compounds, a bis-pivaloyloxymethyl (bisPOM) strategy is especially useful since such compounds are quite stable in buffer and plasma and they are readily transformed to free phosphate inside various cell types. The mechanism for degradation of bis(POM) phosphate inside cells has been studied. During the process, two different degradation enzymes are involved: esterase and phosphodiesterase. Thus, after the cell entry, the mask for the phosphate group is removed and the compounds converted to a biologically active form. Methods have been described to introduce the bispivaloyloxymethyl(POM) phosphate triesters. (Hwang, Y.; Cole, P. A., (2004) Efficient synthesis of phosphorylated prodrugs with bis(POM)-phosphoryl chloride, Org Lett, 6, 1555-6. Farquhar, D.; Chen, R.; Khan, S., (1995) 5′-[4-(Pivaloyloxy)-1,3,2-dioxaphosphorinan-2-yl]-2′-deoxy-5-fluorouridine: a membrane-permeating prodrug of 5-fluoro-2′-deoxyuridylic acid (FdUMP), J Med Chem, 38, 488-95. Farquhar, D.; Khan, S.; Srivastva, D. N.; Saunders, P. P., (1994) Synthesis and antitumor evaluation of bis[(pivaloyloxy)methyl] 2′-deoxy-5-fluorouridine 5′-monophosphate (FdUMP): a strategy to introduce nucleotides into cells, J Med Chem, 37, 3902-9.)

However, there remain unstudied areas relating to Pin I, such as possible synthesis of potent inhibitors of Pin1 that may serve as anti-cancer drug lead compounds.

As background, there is also mentioned what previously has been a separate field involving a-keto amides which inhibit several proteases, such as serine proteases and cysteine proteases. See, e.g., U.S. Pat. No. 7,001,907 issued Feb. 21, 2006, U.S. Pat. No. 6,703,368 issued Mar. 9, 2004 and U.S. Pat. No. 6,288,231 issued Sep. 11, 2001 all to Chatterjee et al. (Cephalon, Inc.) for “Peptide-containing α-ketoamide cysteine and serine protease inhibitors”; U.S. Pat. No. 6,150,378 issued Nov. 21, 2000 to Chatterjee et al. (Cephalon) for “Peptidyl-containing α-ketoamide cysteine and serine protease inhibitors.” The α-keto amide functional group has been used in a wide range of enzyme inhibitors to elucidate the inhibition mechanisms of enzymes including PPIases. (Wang, X. J.; Etzkom, F. A., (2006) Peptidyl-Prolyl Isomerase Inhibitors, Biopolymers: Peptide Science, 84, 125-146.) See U.S. Pat. No. 6,781,000 issued Aug. 24, 2004 and U.S. Pat. No. 6,075,150 issued Jun. 13, 2000 both to Wang, et al. (CV Therapeutics) for “α-ketoamide inhibitors of 20S proteasome”; U.S. Pat. No. 6,774,212 issued Aug. 10, 2004 to Han et al. (Bristol-Meyers Squibb Pharm) for “α-ketoamide inhibitors of hepatitis C virus NS3 protease”; U.S. Pat. No. 6,310,057 issued Oct.30, 2001 and U.S. Pat. No. 6,096,778 issued Aug. 1, 2000 both to Chatterjee et al. (Cephalon) for “α-ketoamide multicatalytic protease inhibitors”; U.S. Pat. No. 6,083,944 issued Jul. 4, 2000 to Chatterjee et al. for “Quinoline-containing α-ketoamide cysteine and serine protease inhibitors”; U.S. Pat. No. 5,670,479 issued Sep. 23, 1997 and U.S. Pat. No. 5,656,600 issued Aug. 12, 1997 to Abelman et al. (Corvas), for “α-ketoamide derivatives as inhibitors of thrombosis”.

Other examples of cc-ketoamides and synthesis thereof include, e.g., U.S. Pat. No. 7,022,738 issued Apr. 4, 2006 to Nakamura et al. (Senju Pharmaceutical Co.) for “α-ketoamide derivatibves and use thereof”; U.S. Pat. No. 6,452,050 issued Sep. 17, 2002 and U.S. Pat. No. 6,143,931 issued Nov. 7, 2000 both to Baldino et al. (ArQule, Inc.) for “Synthesis and use of α-ketoamide derivatives and arrays”; U.S. Pat. No. 6,187,905 issued Feb. 13, 2001 to Hurst et al. for “α-ketoamide derivatives”; U.S. Pat. No. 5,414,122 issued May 9, 1995 to Murabayashi et al. (Shionogi & Co.) for “Process for producing α-ketoamide derivative.”

### SUMMARY OF THE INVENTION

It has now been recognized that α-keto amides can function as Pin1 inhibitors. In addition, families of new Pin1 inhibitors which are α-keto amides (such as, e.g., α-keto amide Ser-Pro analogues) have been synthesized. In particular, α-keto amides are shown to have an activity as transition state mimetics. This functionality permits stable inhibition of catalytic activity. The exemplary compounds described mimic a transition-state “twisted amide”.

Exemplary α-ketoamide compounds within the practice of this invention include: 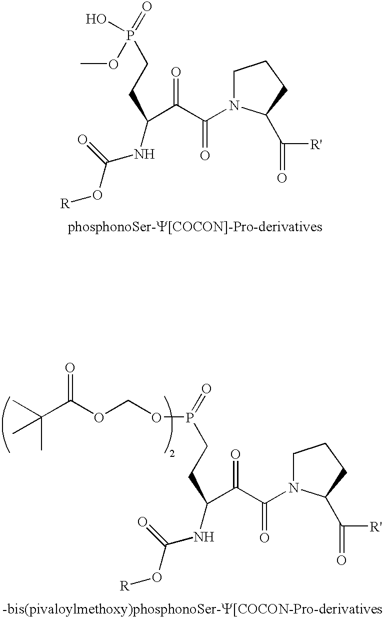 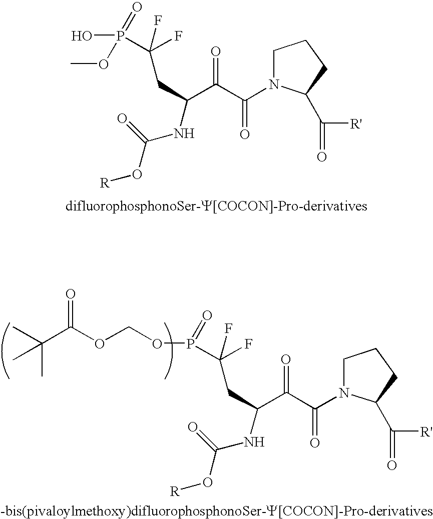
wherein R is a carbonyl group attached to the amine as an amide, and R′ is an amine attached to the carbonyl as an amide. There are a wide variety of R and R′ moieties which can be used within the practice of the invention. In an exemplary embodiment of the invention, the (α-ketoamides are incorporated into peptide structures to serve as mimics. In a particular application, an α-ketoamide Ser-Pro dipeptide analogue into the pentapeptide analogue Ac-Phe-Tyr-pSer-Pro-Arg-NH₂ which is an inhibitor of Pin1 (i.e., the “pSer-Pro” constitutes the α-ketoamide Ser-Pro dipeptide analogue of this invention). Preferably, the peptide mimics of this invention will have one or more α-ketoamide dipeptides incorporated into peptides of 1-20 amino acids in length.

### DETAILED DESCRIPTION OF THE PREFERRED EMBODIMENTS OF THE INVENTION

Because α-ketoamides have an electron-deficient carbonyl group due to the effect of the neighboring electron-withdrawing amide group, the α-ketoamides can serve as transition state mimetics and allow inhibition of catalytic activity. It is now recognized that such an activity for α-ketoamides may be applied in the context of Pin1. This activity has previously been known in a non-Pin1 context; namely, this activity occurs for serine or cysteine proteases through the formation of a tetrahedral intermediate (gem-diol or hemiketal) with the enzyme upon binding. (Maryanoff, B. E., Qiu, X., Padmanabhan, K. P., Tulinsky, A., Almond, H. R., Jr., Patricia, A. G., Greco, M. N., Kaufftnan, J. A., Nicolaou, K. C., Liu, A., Brungs, P. H., Fusetani, N., Molecular basis for the inhibition of human α-thrombin by the macrocyclic peptide cyclotheonamide A, Proc. Natl. Acad. Sci. USA 1993, 90, 8048-8052.)

The property of α-ketoamides mimicking a “twisted amide” transition-state structure with a dihedral angle of 95 to 100 degrees between carbonyl groups, may be exploited in a Pin1 context. A “twisted amide” transition-state structure has previously been observed in a non-Pin1 context. (Rosen, M. K.; Standaert, R. F.; Galat, A.; Nakatsuka, M.; Schreiber, S. L., Inhibition of FKBP rotamase activity by immunosuppressant FK506: twisted amide surrogate. Science 1990, 248, (4957), 863-866.)

An example of making a peptidomimetic according to the invention is, e.g., to incorporate an α-ketoamide into another molecule (preferably a molecule which is a peptide inhibitor of Pin1), such as, e.g., incorporating an α-ketoamide Ser-Pro dipeptide analogue (such as presented in structure 11 below) into a pentapeptide analogue Ac-Phe-Tyr-pSer-Pro-Arg-NH₂ where “pSer-Pro” is the structure presented in structure 11 of Scheme 8 or, for example, the phosphate triesters set forth in Scheme 9 below. The pentapeptide analogue Ac-Phe-Tyr-pSer-Pro-Arg-NH₂ is considered an optimal peptide inhibitor of Pin1. (Zhang, Y.; Fussel, S.; Reimer, U.; Schutkowski, M.; Fischer, G., Substrate-Based Design of Reversible Pin1 Inhibitors. Biochemistry 2002, 41, 11868-11977.) This peptidomimetic constructed by incorporating an α-ketoamide Ser-Pro dipeptide analogue into a pentapeptide analogue Ac-Phe-Tyr-pSer-Pro-Arg-NH₂ is designed as a potential transition-state analogue inhibitor of Pin1, because the α-ketoamide may react with Cys113 in the active site of Pin1 to form the tetrahedral intermediate hemithioketal, or it may act as a surrogate of the “twisted” amide transition state of peptides bound to Pin1. However, it should be understood that the α-ketoamide Ser-Pro dipeptide could be incorporated into any peptide moiety for some applications of the invention and that there could be, for example, one to ten amino acids on either side of the α-ketoamide Ser-Pro dipeptide. Likewise, the invention contemplates the incorporation of other α-ketoamide dipeptides into peptides of 1-20 amino acids in length.

α-ketoamides used in this invention may be synthesized according to known methods, such as, e.g., cyano ylide coupling methodology, synthesis from carbamoylsilane and acid chlorides, transition-metal-catalyzed double carbonylation, amidation of α-keto acids with amines, oxidation from a-hydroxyl amides, and Grignard reagent nucleophilic addition.

For synthesis of dipeptide α-keto amides for use in the present invention, two preferred methods are set forth as follows with reference to Schemes 1-3.

1. Cyano Ylide Coupling Methodology

Wasserman et al. developed versatile strategies for the formation of α-keto amides through the use of the (cyanomethylene)triphenylphosphorane. (Wasserman, H. H.; Ho, W. B., (Cyanomethylene)phosphoranes as Novel Carbonyl 1, 1-Dipole synthons: An Efficient Synthesis of alpha-Keto Acids, Esters, and Amides. J Org. Chem. 1994, 59, 4364-4366.) The reaction is shown in Scheme 1. 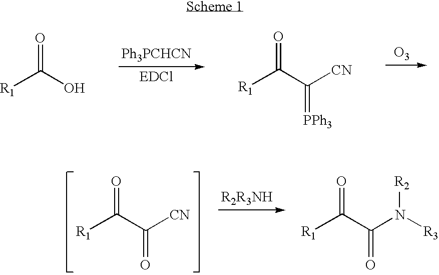
First the carboxylic acid is coupled with (cyanomethylene)triphenylphosphorane in the presence of EDCI and DMAP, then the ylide formed is treated with ozone to generate an α,β-diketo nitrile, which is not stable at room temperature, treated in situ with amines to form α-keto amides. This method has wide application and has been used in synthesis of natural products such us eurystatin A and B, poststatin and verongamine. (Wasserman, supra; Kim, H.-O.; Nakanishi, H.; Lee, M. S.; Kahn, M., Design and Synthesis of Novel Conformationally Restricted Peptide Secondary Structure Mimetics. Org. Lett. 2000, 2, (3), 301-302; Wasserman, H. H.; Wang, J., Synthesis of the Marine Metabolites Verongamine, Hemibastadin-2, and Aerothionin Using the Cyano Ylide Coupling Methodology. J Org. Chem. 1998, 63, 5581-5586.)

This cyano ylide method has been applied to various carboxylic acids, especially amino acids in excellent yields for the first step and in moderate yields for the second step. The advantage of this method is that the synthesis route is short and starting materials are commercially available.

2. From α-Hydroxy Acids

2.1. The Synthesis of α-Hydroxy Acids

Numerous methods to synthesize α-hydroxy amides are known to those in the art. The most common method is through the coupling of α-hydroxy acids with amines. The following Scheme 2 shows a common method used in many dipeptide α-ketoamides syntheses:

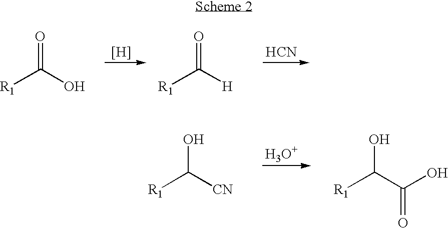

2.2. The Oxidation of α-Hydroxy Amides

Oxidation of α-hydroxy amides is widely used in the synthesis of α-keto amide inhibitors of enzymes. (Zhang, Y.; Fussel, S., Reimer, U., Schutkowski, M., Fischer, G., Substrate-Based Design of Reversible Pin1 Inhibitors, Biochemistry, 2002, 41, 11868-11877). Examples of oxidants include, e.g., Dess-Martin, TEMPO or PDC oxidation.

The oxidation of α-hydroxy amides is a common method used in peptide α-keto amide synthesis; however the synthetic route to the α-hydroxy acids is long. The drawback of this method in the oxidation step is the epimerization of the chiral center adjacent to the ketone group which causes the formation of a mixture of diastereomers. Harbeson et al. reported an oxidation by TEMPO/hypochlorite without epimerization. (Harbeson, S. L.; Abelleira, S. M.; Akiyama, A.; Barrett, R.; Carroll, R. M.; Straub, J. A.; Tkacz, J. N.; Wu, C.; Musso, G. F., Stereospecific Synthesis of Peptidyl α-Keto Amides as Inhibitors of Calpain. J Med. Chem. 1994, 37, 2918-2929.) The general equation of this reaction is shown in Scheme 3.

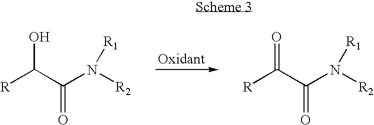

As between the two above-described synthetic methods of α-ketoamides, the cyano ylide methodology is preferred for synthesis in the present invention because only two steps are required for the key dipeptide α-ketoamide syhthesis.

Pin1 inhibitors, such as the inventive Pin1 inhibitors which are peptidomimetics, may be used as anti-cancer agents alone or in conjunction with apoptosis inducers. Pin1 inhibitors (such as the inventive Pin1 inhibitors) may also be useful in the treatment of cocaine addiction. Inhibition of Pin1 may be important in other disease processes because Pin1 is an essential regulator of the G2 to M transition in the cell cycle.

Advantageously, the inventive compounds which are Pin1 inhibitors or which are peptidomimetics should bind more specifically than juglone which is currently used as a Pin1 inhibitor.

Unlike conventional cell cycle inhibitors, which target kinases or phosphatases (of which there are many therefore making specificity difficult to attain), the inventive peptidomimetic Pin1 inhibitors operate by an entirely different mechanism and therefore make possible the specific targeting of cancer cells.

The invention may be further appreciated with reference to the following examples, without the invention being limited to the Examples.

### EXAMPLE 1

The reaction scheme for the synthesis of the α-ketoamide Ala-Pro dipeptide analogue is shown in Scheme 4, which is a model reaction for synthesis of the α-ketoamide Ser-Pro dipeptide analogue. Additionally, the α-ketoamide Ala-Pro dipeptide analogue may be a potential inhibitor of cyclophilin.

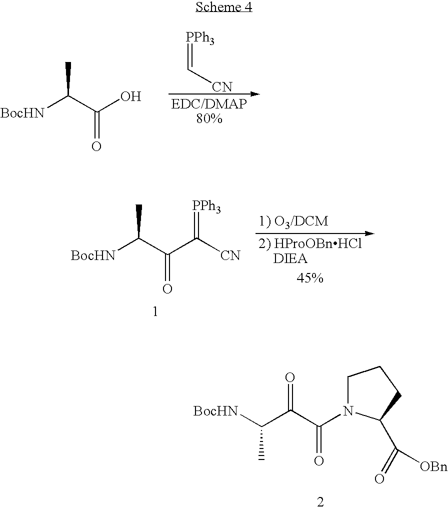

In the first step, the ylide 1 was synthesized from a coupling reaction between commercially available (cyanomethylene)triphenylphosphorane and Boc-L-AlaOH with EDC in the presence of 4-dimethylaminopyridine. Under these conditions, a good yield of 80% was obtained.

In the second step, the ylide 1 was oxidized to an α,β-diketonitrile. This labile electrophile then was reacted in situ with HProOBn to form the α-keto amide 2. From NMR and MS spectra, this α-ketoamide 2 appeared to be obtained as a mixture of isomers in 45% yield.

### EXAMPLE 2

The ozonolysis method of Example 1 was used in the synthesis of α-keto amide Ser-Pro dipeptide analogue (Scheme 5).

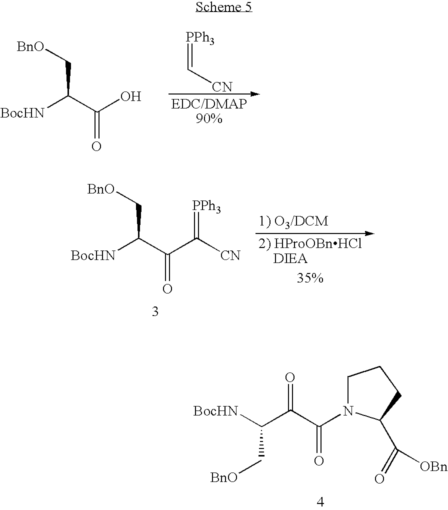

The ylide 3 was synthesized in an excellent yield of 90% by the same method as ylide 1. The Ser-Pro α-ketoamide 4 was obtained as a mixture of isomers in 35% yield. During purification of compound 4 on column, some decomposition happened, making purification of the crude product mixture difficult. For compound 4, at least four steps (hydrogenolysis, deprotection of Boc, protection with Fmoc and phosphorylation) were needed to prepare the phosphorylated FmocSer-Pro α-ketoamide 7 ready for solid phase synthesis of peptides.

Because the Fmoc protecting group is tolerant of ozonolysis according to the literature, FmocSer(Ot-Butyl)OH is selected as the starting material. The ylide 5 thus formed gives the α-ketoamide after ozonolysis and treatment with HProOt-Bu. Two steps are therefore saved in the synthesis of α-ketoamide 7. The ylide 5 has been prepared in 85% yield and α-ketoamide 6 has been prepared in 35% yield (Scheme 6). From the NMR, the compound 6 seemed to be obtained as isomers. It is difficult to isolate them, and they seem to isomerize or decompose on column or in solution. Then the mixture was directly used in the next deprotection step.

The compound 7 characterized by MS was obtained in 30% yield. The product was found to be unstable in solution, and it totally decomposed in CDCl₃ solution after 2 days. The decomposition product appears to be a cyclic dimer (bis-lactone) of 7.

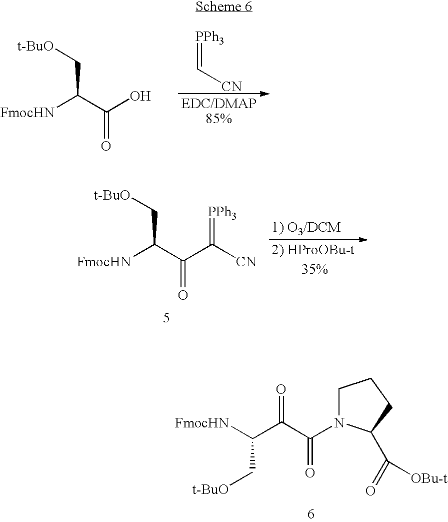

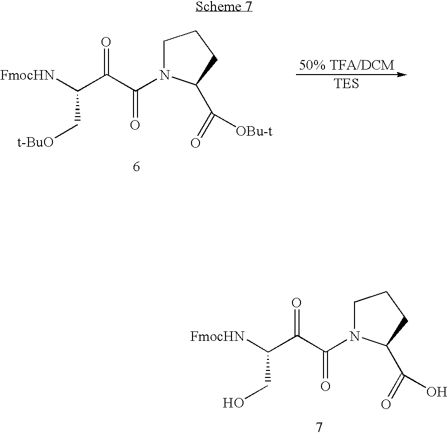

### EXAMPLE 3

For assaying the inhibitory activities of the α-ketoamide inhibitor, Pin1 enzyme is used according to Scheme 8.

A series of different amines (see FIG. 1) and carboxylic acids (see FIG. 2) are coupled to the C-terminus and N-terminus to produce compounds of the type 10. In addition, cell permeable derivatives 11 are prepared to improve bioavailability.

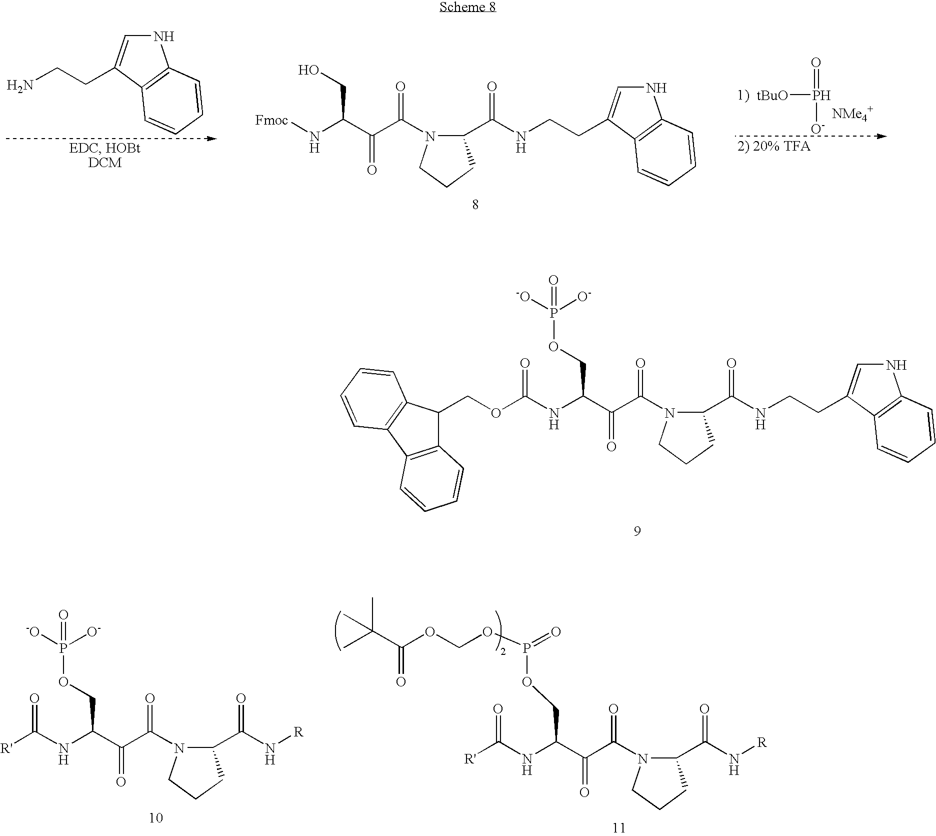

Phosphate monoesters such as those used in the previous schemes are suitable for in vitro enzyme inhibition studies. However, problems with membrane permeability for phosphate esters are well known and reflected in the need for DMSO in cell-based assays conducted with Pin1 inhibitors. (Wang, X. J.; Xu, B.; Mullins, A. B.; Neiler, F. K.; Etzkom, F. A., (2004) Conformationally Locked Isostere of PhosphoSer-cis-Pro Inhibits Pin1 23-Fold Better than PhosphoSer-trans-Pro Isostere, J. Am. Chem. Soc., 126, 15533-15542.) Towards this end, to solve the problems of bioavailability conferred by the specificity of Pin1 for phosphoSer/Thr preceding Pro, using bis-pivaloylmethoxy (POM) phosphate triesters as prodrugs of Pin1 inhibitors is preferred.

The membrane permeability problem has been solved by masking the phosphate with hydrophobic esters that are removed by cellular enzymes, a prodrug strategy. (Wagner, C. R.; Iyer, V. V.; Mclntee, E. J., (2000) J. Med. Res. Rev., 20, 417-451.) Borch has developed indolequinone ester prodrugs of phosphoramidates that are membrane permeable. (Hemick, M.; Borch, R. F., (2003) Studies on the Mechanisms of Activation of Indolequinone Phosphoramidate Prodrugs, J. Med. Chem., 46, 148-154. Hemick, M.; Flader, C.; Borch, R. F., (2002) Design, synthesis and biological evaluation of indolequinone phosphoramidate prodrugs targeted to DT-diaphorase, J. Med. Chem., 45, 3540-3548.) The indene moiety was found to be activated by reduction by NAD(P)H: quinone oxidoreductase (NQO1). (Segnitz, B.; Gehring, U., (1997) The function of steroid hormone receptors is inhibited by the hsp90-specific compound geldanamycin, J Biol Chem, 272, 18694-701. Ross, D.; Siegel, D.; Beall, H.; Prakash, A. S.; Mulcahy, R. T.; Gibson, N. W., (1993) DT-diaphorase in activation and detoxification of quinones. Biorductive activation of Mitomycin C. (Review). Cancer Metastasis Rev., 12, 83-101. Gutierrez, P. L., (2000) The role of NAD(P)H oxidoreductase (DT-diaphorase) in the bioactivation of quinone-containing antitumor agents: A review, Free Radical Biol. Med., 29, 263-275.) pivaloylmethoxymethyl (POM) ester group has been shown to be stable under aqueous conditions in the pH range from 1-8, but it is hydrolyzed by general carboxylate esterases in cells. Two POM groups on a phosphate generally confer membrane permeability and biological activity on a number of different types of phosphates. (Hwang, Y.; Cole, P. A., (2004) Efficient synthesis of phosphorylated prodrugs with bis(POM)-phosphoryl chloride, Org Lett, 6, 1555-6.) The second strategy is preferred to improve the bioavailability of Pin1 ligands, using the reagent synthesized by Hwang et al. to phosphorylate lead compounds (Scheme 9).

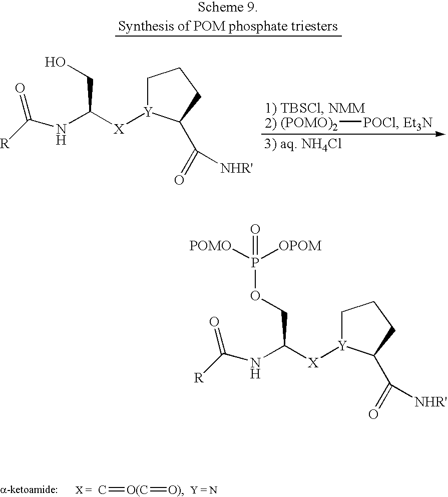

### EXAMPLE 4

Experimentation was conducted as follows.

General. Flash chromatography was performed on 230-400 mesh, ASTM silica gel with reagent grade solvents. NMR spectra were obtained at ambient temperature in CDC13 unless otherwise noted. Proton and carbon-13 NMR spectra were obtained at 500 and 125 MHz, respectively.

Compound 4: Ozone was added to the solution containing ylide 3 (240 mg, 0.41 mmol) until the pale green color stayed unchanged under −78 C. Extra Ozone was purged by Nitrogen. HProOBn.HCl (110 mg, 0.46 mmol) in 1 ml DCM containing 63.9 mg DIEA (0.49 mmol) was cooled and added to the reaction mixture slowly. The resulting mixture was stirred at −78 C. for 2 hours then at room temperature for 1 h. The reaction mixture was diluted with 20 mL DCM and then washed with Brine twice. The organic layer was dried over Na₂SO₄. After filtration and evaporation, the residue was purified on chromatography (eluent: 30% EA in Hexane) to give the ketoamide 4 as the mixture of isomers (73 mg, crude yield: 33%). HNMR: δ 1.40 (m, 9H), δ 1.83-2.15 (4H), 3.50-3.75 (m, 2H), δ 4.05 (m, 1H), δ 4.38-4.55 (m, 4H), δ 5.15 (3H), 5.40 (d, 1H), δ 7.35 (10H). CNMR: 196.8, 171.8, 169.5, 155.4, 138.0, 135.8, 128.6-128.4, 80.0, 73.3, 71.0, 66.9, 59.2, 52.1, 47.2, 32.0, 29.0, 24.7. LRMS: calcd for C28H34N207 (MH+) m/z=511.2 found m/z=511.4.

Compound 5: A solution of Fmoc-L-SerOH (648 mg, 1.69 mmol), (cyanomethylene)phosphorane (509 mg, 1.69 mmol), DMAP (62 mg, 0.51 mmol) and EDC (387 mg, 2.03 mmol) in CH₂Cl₂ (10 mL) was stirred at rt for 3 h. The reaction mixture was then diluted with CH₂Cl₂ (20 mL), washed with water (30 mL×2) and Brine (30 mL). The organic layer was dried over Na₂SO₄, filtered and evaporated under reduced pressure. The crude product was purified by chromatography on silica (gradient: 50 to 60% EtOAc in hexane) to yield the ylide 5 (960 mg, 85.2%) as an oil. H NMR: δ 1.21 (s, 9H), 3.80 (q, 1H), 4.18 (t, 2H), 4.27 (t, 1H), 4.36 (t, 1H), 5.00 (d, 1H), 5.98(d, 2H), 7.25-7.75 (m, 23H).

Compound 6: Ozone was added to the solution containing ylide 5 (172 mg, 0.26 mmol) until the pale green color stayed unchanged under −78 C. Extra Ozone was purged by Nitrogen. HProOt-Bu (44 mg, 0.26 mmol) in 1 ml DCM was cooled and added to the reaction mixture slowly. The resulting mixture was stirred at −78 C. for 2 hours then at room temperature for 1 h. The reaction mixture was diluted with 20 mL DCM and then washed with Brine twice. The organic layer was dried over Na₂SO₄. After filtration and evaporation, the residue was purified on chromatography (eluent: 30% EA in Hexane) to give the ketoamide 6 as the mixture of isomers (50 mg, crude yield: 33%). HNMR: δ 1.12 (d, 9H), 1.45 (m, 9H), δ 1.85-2.25 (m, 4H), 3.68 (m, 2H), δ 4.15 (m, 1H), δ4.25 (m, 1H), 4.38 (m, 2H), δ 5.22 (1H), 5.70 (m, 1H), δ 7.28-7.75 (8H). CNMR: 195.7, 170.2, 161.7, 155.9, 144.1, 141.4, 127.8, 127.1, 125.2, 120.0, 82.0, 67.2, 60.0, 58.4, 47.7, 47.2, 29.0, 28.0, 21.8. LRMS: calcd for C21H28N206 (MH+) m/z =565.67 found m/z=565 (EI+).

Compound 8: Compound 6 (140 mg, 0.248 mmol) was dissolved in 6 mL DCM, and then 3 mL TFA and 0.01 mL TES were added. The resulting reaction mixture was stirred at room temperature for 3 hours. After evaporation, the residue was purified by column (eluent: CHCl₃:CH₃OH=20:1 or MeOH:CH₂Cl₂=1:8). LRMS: calcd for C24H24N207 (MH+) m/z=453.46 found m/z=453.17.

Another preferred embodiment of the invention provides a phosphate mimic modified compound comprising an α-ketoamide compound (such as, e.g., any of the above-mentioned α-ketoamide compounds) modified with at least one phosphate mimic (such as, e.g., phosphonate, difluorophosphonate, and bis(pivaloylmethoxy) mimics), such as, e.g., the following phosphate mimic modified compounds: 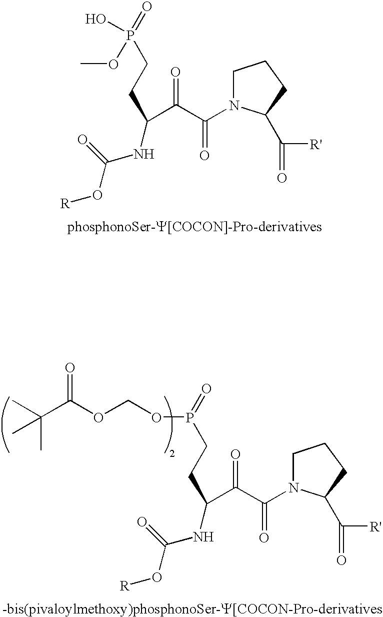 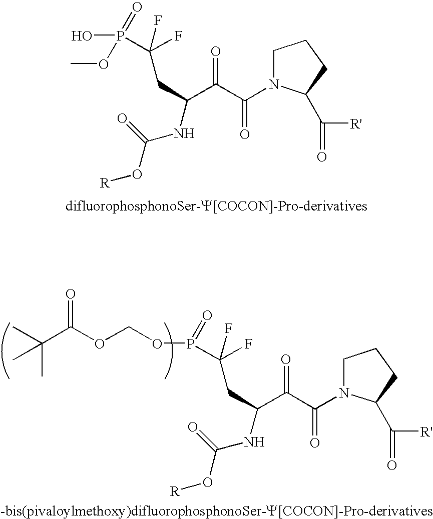
wherein R is a carbonyl group attached to the amine as an amide, and R′ is an amine attached to the carbonyl as an amide.

In all the above formulae where R has been mentioned, preferred examples of R are, e.g., the following 26 acid and acid chloride synthons, where the connection to the c-ketoamide molecule is made through the carboxylic acid moiety.

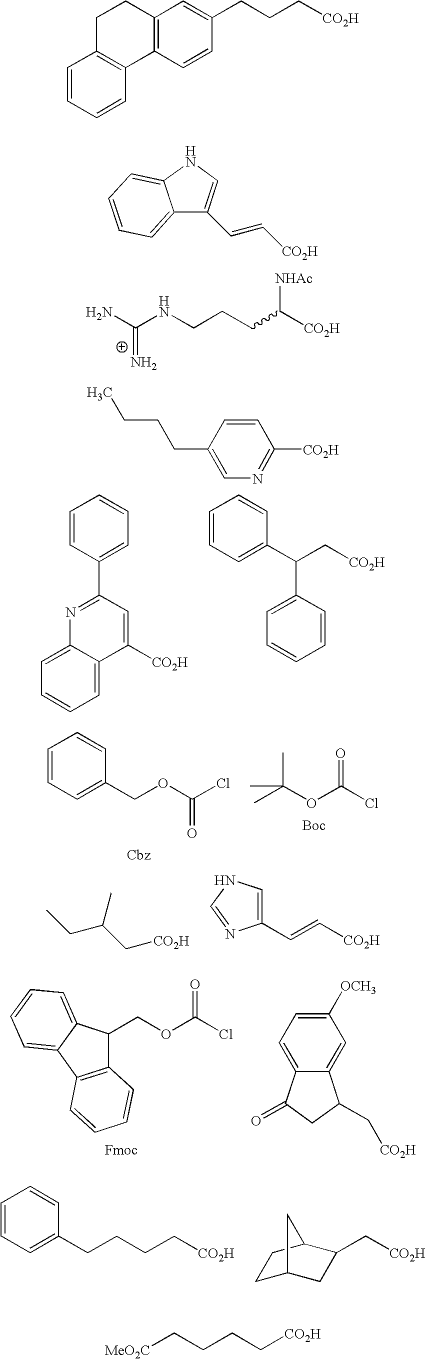

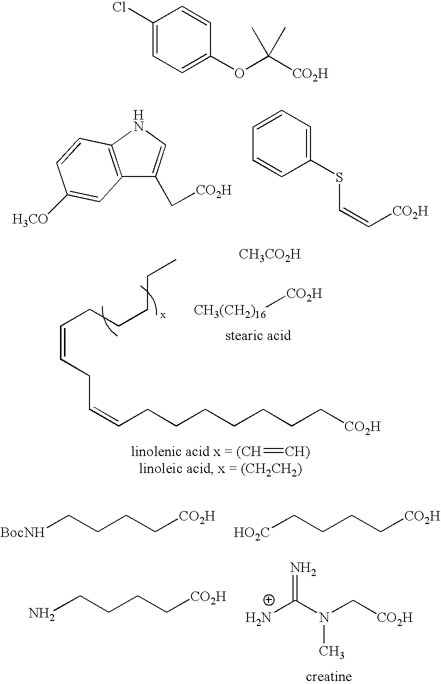

In all the above formulae where R′ has been mentioned, preferred examples of R′ are, e.g., the following 30 amine synthons, where the connection to the α-ketoamide molecule is made through the amine moiety:

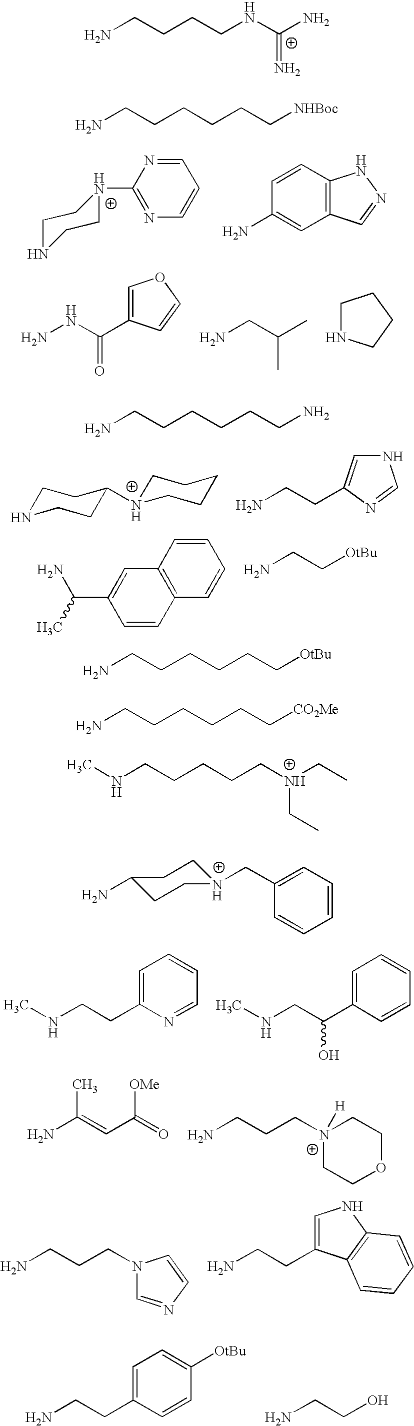

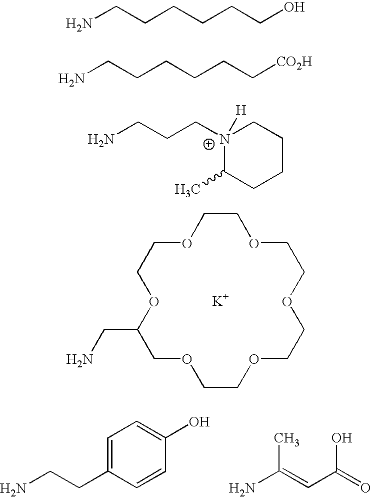

As discussed in more detail above, the R and R′ moiety can be the same or different, and can each be 1-20 amino acids in length. That is, a generalized formula for a peptide, which can function as a mimic or for other purposes, according to the invention would be R-α-ketoamide-R′, where R is connected at the carbonyl end of the amino acid sequence and R′ is connected at the amino end of the amino acid sequence. In a number of embodiments, the “α-ketoamide” will have a phosphorous moiety (e.g., see schemes 8 and 9 above).

While the invention has been described in terms of its preferred embodiments, those skilled in the art will recognize that the invention can be practiced with modification within the spirit and scope of the appended claims.

What is claimed is:

1. A peptidomimetic compound comprising an α-ketoamide, wherein the peptidomimetic compound has anti-Pin1 activity.

2. The peptidomimetic compound of claim 1, wherein the α-ketoamide is α-ketoamide Ser-Pro dipeptide analogue.

3. The peptidomimetic compound of claim 2 wherein said compound is a pentapeptide.

4. A peptide compound having the general structural formula
R-α-ketoamide-R′
where R and R′ can be the same or different, and each includes 1-20 amino acids.

5. The peptide compound of claim 4 wherein said α-ketoamide moiety is based on a Ser-Pro dipeptide.

6. The peptide compound of claim 4 wherein said α-ketoamide compound is modified with a phosphorus moiety.

7. An α-ketoamide compound, wherein the α-ketoamide compound is selected from the group consisting of: 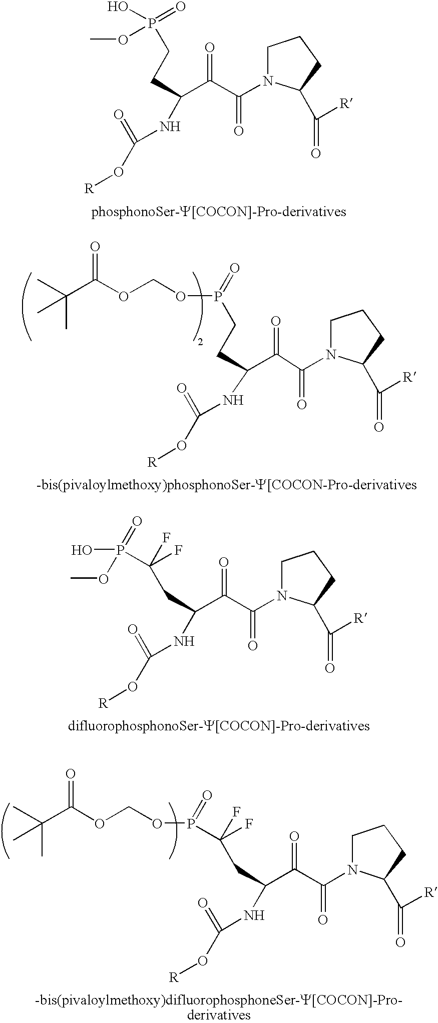
wherein R includes a carbonyl group attached to the amine as an amide, and R′ includes an amine attached to the carbonyl as an amide.

8. The α-ketoamide compound of claim 7 where each of R and R′ may be the same or different and each of R and R′ include one to twenty amino acids.

9. The α-ketoamide compound of claim 7, wherein R is selected from the group consisting of the following 26 acid and acid chloride synthons where the connection to the α-ketoamide molecule is made through the carboxylic acid moiety:

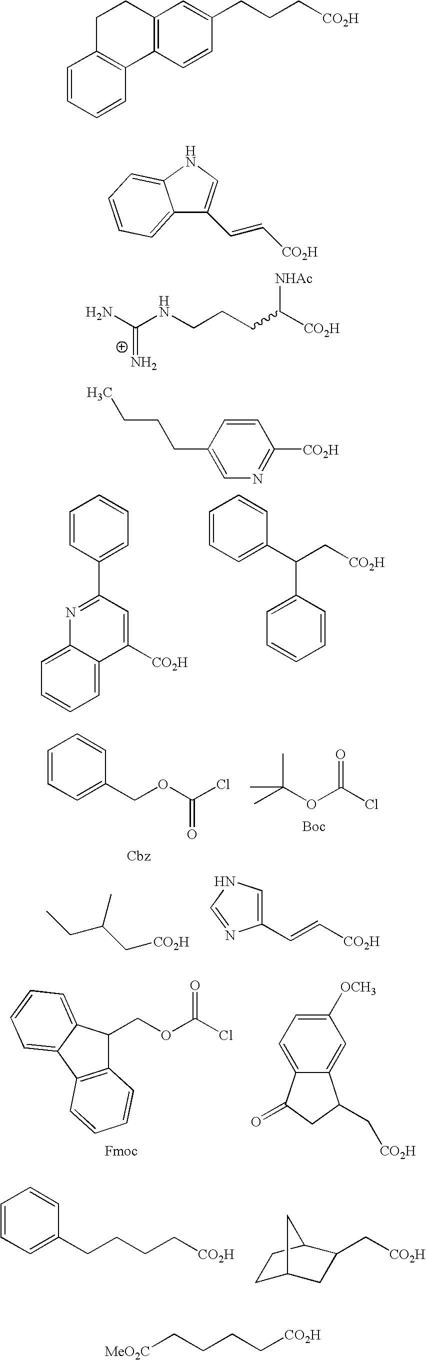

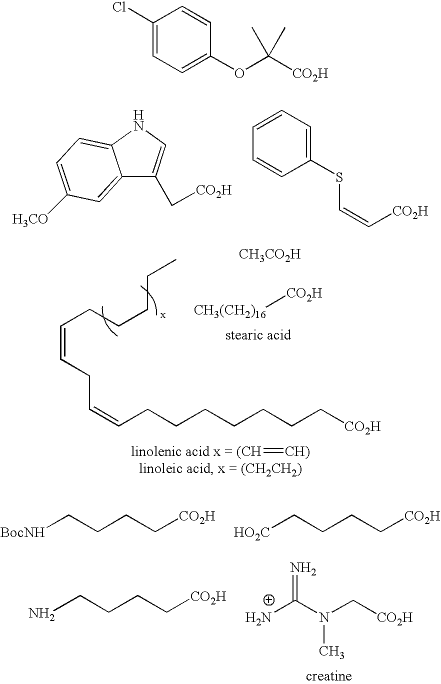

10. The α-ketoamide compound of claim 7, wherein R′ is selected from the group consisting of the following 30 amine synthons where the connection to the α-ketoamide molecule is made through the amine moiety:

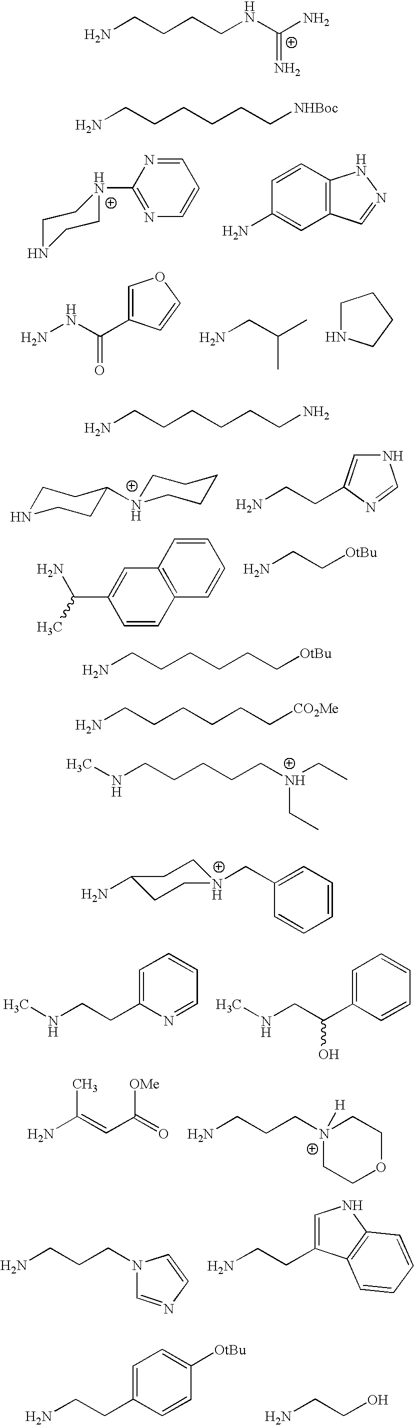

11. A peptidomimetic compound containing an α-ketoamide moiety selected from the group consisting of α-ketoamide phosphoSer-Pro, phosphoThr-Pro or Glu-Pro and the corresponding α-ketoamide phospho(D)Ser-Pro, phospho(D)Thr-Pro, and phospho(D)Glu-Pro dipeptide analogue.

12. A compound comprising an αc-ketoamide and a peptide analogue, wherein the α-ketoamides is one or both of: (1) a transition state mimetic and (2) mimicks a transition-state “twisted amide.”

13. The compound of claim 12, wherein the α-ketoamide is α-ketoamide Ser-Pro dipeptide analogue.

14. A method of synthesizing a peptidomimetic, comprising: incorporating an α-ketoamide into a molecule, wherein a peptidomimetic is constructed.

15. The method of claim 14, wherein the α-ketoamide is an α-ketoamide Ser-Pro dipeptide analogue.

16. The method of claim 14, wherein the molecule is a pentapeptide analogue Ac-Phe-Tyr-pSer-Pro-Arg-NH₂.
